(19) United States
(12) Patent Application Publication
(10) Pub. No.: US 2025/0000633 A1
(43) Pub. Date: Jan. 2, 2025

(54) 3D AUXETIC STRUCTURES AND FABRICATION METHODS THEREOF

(71) Applicants: GEORGIA TECH RESEARCH CORPORATION, Atlanta, GA (US); EMORY UNIVERSITY, Atlanta, GA (US)

(72) Inventors: Jeong Hun PARK, Atlanta, GA (US); Scott J. HOLLISTER, Atlanta, GA (US); Sarah Jo TUCKER, Atlanta, GA (US); Lizhen WANG, Atlanta, GA (US); Hyun-Ji PARK, Atlanta, GA (US); Michael E. DAVIS, Atlanta, GA (US); Sarah K. RUTLEDGE, Atlanta, GA (US); Adam S. VERGA, Atlanta, GA (US)

(21) Appl. No.: 18/698,693

(22) PCT Filed: Oct. 6, 2022
(86) PCT No.: PCT/US2022/045937

§ 371 Date: Apr. 4, 2024

Related U.S. Application Data
(60) Provisional application No. 63/252,742, filed on Oct. 6, 2021.

(51) Int. Cl. A61F 2/00; B29C 64/153; B29K 105/04; B29L 31/00; B29L 31/40; B33Y 10/00; B33Y 80/00; C12M 1/12

(52) U.S. Cl. CPC A61F 2/0077; B33Y 80/00; C12M 25/14; A61F 2002/0081; A61F 2210/0057; A61F 2230/0013; A61F 2230/0043; A61F 2240/002; A61F 2250/0037; A61F 2250/0067; B29C 64/153; B29K 2105/041; B29K 2995/0082; B29L 2031/40; B29L 2031/7532; B33Y 10/00

(57) ABSTRACT

Disclosed herein is an apparatus comprising: an auxetic unit-cell, each comprising: a first longitudinal beam, a second longitudinal beam fixably attached to the first longitudinal beam at a first hinge region, the second longitudinal beam is orthogonally disposed with respect to the first longitudinal beam; and a third longitudinal beam fixably attached to the first longitudinal beam at a second hinge region, the third longitudinal beam is orthogonally disposed with respect to the first and second longitudinal beams, wherein the first, second and third longitudinal beams all are different lengths, wherein the first and second hinge region, and the first and third longitudinal beam are configured to bend under load applied in a direction non-parallel to the longitudinal axis of the first longitudinal beam.

### RELATED APPLICATION

This PCT application claims priority to, and the benefit of, U.S. Provisional Patent Application No. 63/252,742, filed Oct. 6, 2021, entitled “Bioabsorbable 3D Auxetic Implants and Fabrication Methods Thereof,” which is incorporated by reference here in its entirety.

### GOVERNMENT SUPPORT CLAUSE

This invention was made with government support under Grant No. GR10003058, awarded by the National Institutes of Health (NIH). The government has certain rights in the invention.

### BACKGROUND

Tissue engineering has been defined as an interdisciplinary field that applies the principles of engineering and life sciences toward the development of biological substitutes that restore, maintain, or improve tissue function. Three general strategies are employed in tissue engineering: use of isolated cells or cell substitutes, use of tissue-inducing substances, and use of cells placed on or within matrices.

Cells are often implanted or “seeded” into an artificial structure capable of supporting three-dimensional tissue formation. These structures, typically called “scaffolds,” are often critical, both ex vivo as well as in vivo, to provide cells with appropriate microenvironments. Scaffolds serve one or more of the following purposes: allow cell attachment and migration; deliver and retain cells and biochemical factors; enable diffusion of vital cell nutrients and expressed products; and exert certain mechanical and biological influences to modify the behavior of the cell structure.

To achieve the goal of tissue reconstruction, scaffolds must meet specific requirements. A high porosity and an adequate pore size are necessary to facilitate cell seeding and diffusion throughout the whole structure of both cells and nutrients. A porous tissue scaffold (construct) should be sufficiently robust to accommodate the forces applied by cells and other outboard mechanical loads imposed during wound healing, blood flow, and patient activity. A scaffold's elastic properties are critical to its efficacy in regenerating tissue and reducing inflammatory responses and must be matched with the elastic properties of native tissue. The elastic behavior of a porous scaffold can be described by its elastic modulus and Poisson's ratio, which depend on its porosity, the properties of the biomaterial making up the rib structures, and any anisotropic behavior due to the presence of pores. Optimizing these attributes requires control over pore size and geometry with the restriction of arranging the pores, so they are open to the environment and completely interconnected.

Although yield strength and stiffness (elastic modulus) are of vital importance in providing the scaffold with satisfactory mechanical integrity and show power-law behavior with regard to porosity, it does not fully characterize a construct's elastic behavior since it only describes deformation in the loading direction and does not address deformations in the transverse direction.

Poisson's ratio, on the other hand, describes the degree to which a material can contract (expands) transversally when axially strained and is the property that addresses transverse strains resulting from axial deformations. The Poisson's ratio of virtually every porous biomaterial tissue construct is positive, i.e., it contracts in the transverse direction upon expanding in the axial direction. In some applications, scaffolds having a negative Poisson's ratio may be more suitable for emulating the behavior of native tissues and accommodating and transmitting forces to the host tissue site.

When Poisson's ratio is negative, expansion occurs in both the axial and transverse directions simultaneously. This unusual phenomenon has been shown to occur in crystalline materials such as crystalline α-cristobalite SiO₂, materials with hinged crystal structures, carbon allotropes, foams, microporous polymers and laminates, and other extreme states of matter.

It has been shown that man-made auxetic structures can be constructed by patterning non-auxetic polymers with an artificial lattice of rib-containing unit-cells. This sort has been termed as “cellular” or “hinged” materials, owing to the fact that their constitutive pore structure can have a sizable effect on their mechanical behavior. Several unit-cell models have been proposed, each having well-defined strain-dependent Poisson's ratios (Poisson's function) described analytically. In the past, auxetic polyurethane foams have been formed by annealing the foams in a compressed state, which naturally causes a re-organization in their cellular microstructure.

What is needed in the art are 3D auxetic unit-cells in a cube form with a symmetrical arrangement of orthogonally patterned lines, wherein the lines symmetrically arranged on each plane intersect at the points where they are bent at a 90° angle with the lines on surrounding perpendicular planes.

### SUMMARY

Disclosed herein are 3D auxetic structures comprising 3D auxetic unit-cells in a cube or rectangular form with a symmetrical arrangement of orthogonally patterned lines in which the lines are symmetrically arranged on each plane intersect at the points where they bent at a 90° angle with the lines on surrounding perpendicular planes. The 3D auxetic unit-cells include more joints that the lines intersect than prior designs (e.g., re-entrant Honeycomb) in having a symmetrical arrangement of orthogonally patterned lines. Under compression, these joints rotate and induce the bending of the lines between the joints to absorb the compressive load so that the 3D auxetic structure has high initial flexibility. The 3D auxetic unit-cell also has an orthotropic configuration with symmetric mechanical properties that are similar along the X-, Y-, and Z-axis. This mechanical property exhibits cubic symmetry, unlike other, existing 3D auxetics, ensuring the same stiffness to resist variable loading conditions from different directions, which can avoid the risk of unexpected weakness or failure in a particular loading direction.

In an aspect, an apparatus (e.g., tissue reconstruction implant or in vitro tissue model) is disclosed comprising: a 3D auxetic structure formed of a periodic network of orthogonally patterned regions, including a first orthogonally patterned region and a second orthogonally patterned region, the first orthogonally patterned region being coupled to the second orthogonally patterned region, each comprising: a) a first longitudinal beam having a first length, b) a second longitudinal beam fixably attached to the first longitudinal beam at a first hinge region, the second longitudinal beam having a second length and is orthogonally disposed with respect to the first longitudinal beam, wherein the first length is different from the second length; and c) a third longitudinal beam fixably attached to the first longitudinal beam at a second hinge region, the third longitudinal beam having a third length and is orthogonally disposed with respect to the first longitudinal beam and the second longitudinal beam, wherein the third length is different from the first length and second length, wherein the first hinge region, the first longitudinal beam, the second hinge region, and the third longitudinal beam are configured to bend under load applied in a direction non-parallel to the longitudinal axis of the first longitudinal beam. Also disclosed are methods of making and using the apparatus.

In some embodiments, the first orthogonally patterned region and the second orthogonally patterned region each forms an auxetic unit-cell having symmetrical arrangements of orthogonally patterned beams on each orthogonal plane.

In some embodiments, the first hinge region, the first longitudinal beam, the second hinge region, and the third longitudinal beam are configured to bend under the load as a compression load from a bending behavior of the first longitudinal beam and third longitudinal beam when the first hinge region and the second hinge region bend under the same compression load.

In some embodiments, the first longitudinal beam has a constant first cross-section area, wherein the second longitudinal beam has a constant second cross-section area, wherein the third longitudinal beam has a constant third cross-section area, wherein the first cross-section area is the same as the second cross-section area, and wherein the second cross-section area is the same as the third cross-section area.

In some embodiments, the first longitudinal beam has a constant first cross-section area, wherein the second longitudinal beam has a constant second cross-section area, wherein the third longitudinal beam has a constant third cross-section area, wherein the first cross-section area is the same as the second cross-section area, and wherein the second cross-section area is different from the third cross-section area.

In some embodiments, the first longitudinal beam has a constant first cross-section area, wherein the second longitudinal beam has a constant second cross-section area, wherein the third longitudinal beam has a constant third cross-section area, wherein the first cross-section area is different from the second cross-section area, and wherein the first and second cross-section areas are different from the third cross-section area.

In some embodiments, the auxetic structure is a low-density structure having a define-able void fraction between 60% and 95% (e.g., to provide high permeability for cell growth within the auxetic structure).

In some embodiments, the first longitudinal beam of the first orthogonally patterned region has a first end and second end, and wherein the first longitudinal beam of the second orthogonally patterned region is fixably coupled to the first longitudinal beam of the first orthogonally patterned region in between the first end and second end.

In some embodiments, the first longitudinal beam of the first orthogonally patterned region has a first end and second end, and wherein the first longitudinal beam of the second orthogonally patterned region is fixably coupled to the first longitudinal beam of the first orthogonally patterned region at the first end.

In some embodiments, the apparatus further includes a third orthogonally patterned region coupled to the second orthogonally patterned region, the third orthogonally patterned region comprising a fourth longitudinal beam having a fourth length, a fifth longitudinal beam fixably attached to the fourth longitudinal beam at a third hinge region, the fifth longitudinal beam having a fifth length and is orthogonally disposed with respect to the fourth longitudinal beam, wherein the fourth length is different from the fifth length; and a sixth longitudinal beam fixably attached to the fourth longitudinal beam at a fourth hinge region, the sixth longitudinal beam having a sixth length and is orthogonally disposed with respect to the fourth longitudinal beam and the fifth longitudinal beam, wherein the sixth length is different from the fourth length and fifth length, wherein the third hinge region, the fourth longitudinal beam, the fourth hinge region, and the sixth longitudinal beam are configured to bend under the load applied in the direction non-parallel to the axis of the fifth longitudinal beam.

In some embodiments, the third orthogonally patterned region is disposed proximal to the first orthogonally patterned region such that the fourth longitudinal beam of the third orthogonally patterned region is parallel to the first longitudinal beam of the first orthogonally patterned region.

In some embodiments, the auxetic structure comprises a biocompatible material.

In some embodiments, the auxetic structure comprises a polymeric material, for example one or more of the materials: PCL, PGD, PGL, polyurethane, PLLA, PLGA, PGS.

In some embodiments, the auxetic structure comprises a biodegradable (e.g. bioabsorbable) material.

In some embodiments, the apparatus comprises a bioactive material or combination of bioactive materials (e.g., hydrogel-based biomaterial, e.g., comprising collagen, alginate, decellularized extracellular matrix (ECM), gelMA; with or without growth factor reagents and/or therapeutics). In some embodiments, the first longitudinal beam is fabricated with microscale voids.

In some embodiments, the auxetic structure is fabricated via additive manufacturing (e.g., selective laser sintering, extrusion, stereolithography, digital light processing, fused filament fabrication, polyjet).

In some embodiments, the orthogonally patterned regions include a repeating L-shape structure in the first planar direction and a second planar direction orthogonal to the first planar direction.

In some embodiments, the orthogonally patterned regions include a repeating C-shape structure in the first planar direction and a second planar direction orthogonal to the first planar direction.

In some embodiments, the orthogonally patterned regions include a repeating I-shape structure in the first planar direction and a second planar direction orthogonal to the first planar direction.

In some embodiments, the orthogonally patterned regions include a repeating L-shape structure coupled to a repeating C-shape structure in the first planar direction and a second planar direction orthogonal to the first planar direction.

In some embodiments, the apparatus is configured as a tissue or organ implant.

In some embodiments, the apparatus is configured as an implant for one of: breast reconstruction, facial reconstruction, calcaneal fat pad reconstruction, buttock reconstruction, brain tissue defect reconstruction.

In some embodiments, the apparatus is configured for use as an in vitro tissue model.

In some embodiments, the in vitro tissue model comprises hydrogel-based biomaterials incapsulating cardiac cells and relevant bioactive materials, growth factors, or drugs.

In some embodiments, the apparatus is configured as an in vitro model of one of: cardiac tissue, adipose tissue, liver tissue, kidney tissue, neuronal tissue, cartilage tissue.

In some embodiments, the in vitro tissue model comprises one or more of: cardiac cells, (e.g. cardiac myocytes), adipose cells, adipose derived stem cells, hepatocytes, human skeletal muscle cells, induced pluripotent stem cells, chondrocytes.

In some embodiments, the in vitro tissue model is configured for one or more of: biological assessment, detecting and modeling of diseases and disorders, and drug discovery and screening, lab-grown food testing

In some embodiments, the auxetic structure comprises a framework for a hydrogel.

In some embodiments, the apparatus is configured to conform to patient-specific anatomy (e.g. through use of patient image data).

In another aspect, an apparatus is disclosed, comprising: a network of auxetic structure unit-cells configured to be utilized for purposes of adipose tissue reconstruction.

In another aspect, an apparatus is disclosed, comprising: a network of auxetic structure unit-cells configured to be utilized for purposes of breast reconstruction.

In some embodiments, the apparatus, configured to be utilized for purposes of breast reconstruction, is further configured to conform to patient-specific anatomy (e.g. through use of patient image data).

In another aspect, a method is disclosed comprising: forming, via additive manufacturing, the apparatus of any one of the above-discussed claims.

In some embodiments, an apparatus which exists as a computer model for tissue modeling in a virtual environment.

### DESCRIPTION OF DRAWINGS

FIG. 1A-1I show elasto-static and viscous Stokes fluid flow homogenization analysis of auxetic unit-cells according to intervals between parallel lines. a) Structure of the unit-cells with the interval of 0.4 mm (I), 0.8 mm (II), and 1.2 mm (III). b) Comparison of void fraction in auxetic unit-cells according to the three intervals. Effective c) Young's modulus, d) Shear modulus, and e) Poisson's ratio of the unit-cells. Simulation to calculate effective mechanical properties were performed using 200 MPa Young's modulus and 0.3 Poisson's ratio under mixed uniform boundary condition. The errors in orthotropic approximations were 0.03719%, 0.07151%, and 0.1436% for auxetic unit-cells with intervals of 0.4 mm, 0.8 mm, and 1.2 mm, respectively. f) Velocity magnitude of flow through the unit-cells over an ISW boundary condition. The colors indicate the magnitude of the mean velocity, which increases from blue to red. g) Comparison of absolute permeability in auxetic unit-cells. Simulation to calculate the absolute permeability was performed using the Stokes flow boundary conditions of linear pressure variation on side walls (LPVSW), impermeable side walls (ISW), and solid side walls (SSW). The fluid viscosity used in the simulation was 0.001 Pas to approximate the viscosity of DMEM with 10% FBS at 25° C. (0.00094 Pa·s). The auxetic unit-cell was considered an impermeable solid line. h) a detailed view of the auxetic cell showing reference numbers. i) a detailed view of the auxetic unit-cell showing internal structure.

FIG. 2A-2E show design process of the auxetic cube using the auxetic unit-cell assembly of 3×3×3 array. a) A 3D auxetic unit-cell. b) A 3×1 unit-cell array was created by placing the face of one unit-cell in contact with the face of other unit-cells. c) Three of the 3×1 arrays were added in a same way to create 3×3 arrays. d) Three of the 3×3 arrays were added along Z-axis in a same way to create 3×3×3 arrays. e) Protrusions at the edge of an assembly were then cut to create auxetic cubes with all the lines connected, a flat exterior surface, and a specific size.

FIG. 3A-3H show structural and mechanical analysis of auxetic cubes by line intervals. a) Structure of the auxetic cubes with line intervals of 0.4 mm (I), 0.8 mm (II), and 1.2 mm (III). Comparison of b) void fraction and c) compressive modulus of auxetic cubes by intervals. d) Deformation behavior of auxetic cube with 0.4 mm interval at 0% (top), 6% (middle), and 12% (bottom) of Z-axis strain, shown as Phase I (P I), Phase II (P II), and Phase III (P III). The local level of effective stress (von Mises stress) is shown; its increases from blue to green to red. Strain (top), Poisson's ratio (middle), and stress (bottom) curves of auxetic cubes with the interval of e) 0.4 mm, f) 0.8 mm, and g) 1.2 mm. The base material of the auxetic cubes was modeled as a linear isotropic elastic material with Young's modulus of 200 MPa and a Poisson's ratio of 0.3 in simulation under the assumption of large deformation and contact between lines. h) a detailed view of the auxetic cell.

FIG. 4A-4H shows a) Structure of the auxetic cubes with different sizes of 5.6×5.6×5.6 mm (I), 15.8×15.8×15.8 mm (II), 26×26×26 mm (III), and 36.2×36.2×36.2 mm (IV). b) Void fraction comparison of auxetic unit-cells by sizes. c) Comparison of compressive modulus of auxetic cubes by intervals calculated from FE simulation and actual Mechanical tests. VD and HD indicate the vertical and horizontal direction of compression, respectively. d) Deformation behavior of auxetic cube of 26×26×26 mm size at 0% (top), 12% (middle), and 15% (bottom) of Z-axis strain. Yellow, blue, and purple backgrounds indicate Phase I (P I), Phase II (P II), and Phase III (P III), respectively. The colors indicate the local level of effective stress (von Mises stress), its increases from blue to green to red. Stress (top), Poisson's ratio (middle), and Strain (bottom) curves of auxetic cubes with the size of e) 26×26×26 mm, and f) 36.2×36.2×36.2 mm. The base material of the auxetic cubes was modeled as a linear isotropic elastic material with Young's modulus of 200 MPa and Poisson's ratio of 0.3 in simulation under the assumption of large deformation and contact between lines. g) Constructed volume image of auxetic cube single line, including internal microporosity. h) Unit-cubes in different planes.

FIGS. 5A-5D shows structural and viscous Stokes fluid flow homogenization analysis of auxetic cube single lines. Simulation to calculate the absolute permeability of the single lines was performed using the boundary conditions of linear pressure variation on side walls (LPVSW), impermeable side walls (ISW), and solid side walls (SSW). The fluid viscosity used in the simulation was 0.001 Pa's to approximate the viscosity of DMEM with 10% FBS at 25° C. (0.00094 Pa·s). a) Constructed volume image of auxetic cube single line. b) Comparison of void fraction between vertical and horizontal single lines. c) Construction of reverse volume image of a single line. d) Isometric and longitudinal view of streamlines for fluid flow through the single line under a boundary condition of ISW.

FIG. 6A-6B show the example of the patient-specific auxetic implant that has the woman's native breast shape for breast reconstruction. a) Computer-aided design (CAD) model of the auxetic breast implant. b) the fabricated auxetic breast implant using selective laser sintering (SLS) based 3D printing.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed.

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The term “comprising” and variations thereof, as used herein, is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification, the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce,” or “abrogate,” (used interchangeably) or other forms of the word, such as “reducing” or “reduction,” or “abrogating” or “abrogation” is meant lowering of an event or characteristic. It is understood that this is typically in relation to some standard or expected value, in other words, it is relative, but that it is not always necessary for the standard or relative value to be referred to.

By “increase” or other forms of the word, such as “increasing,” is meant raising or elevating. It is understood that this is typically in relation to some standard or expected value, in other words, it is relative, but it is not always necessary for the standard or relative value to be referred to.

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Control” refers to a sample or standard used for comparison with an experimental sample. In some embodiments, the control is a sample obtained from a healthy subject (or a plurality of healthy subjects), such as a subject or subjects not expected or known to have a particular polymorphism. In additional embodiments, the control is a historical control or standard reference value or range of values (such as a previously tested control sample or a plurality of such samples), or group of samples that represent a baseline or normal values. A positive control can be an established standard that is indicative of a specific methylated nucleotide. In some embodiments, a control nucleic acid is one that lacks a particular methylated nucleotide and is used in assays for comparison with a test nucleic acid, to determine if the test nucleic acid includes the methylated nucleotide.

As used herein, “therapeutic” generally can refer to treating, healing, and/or ameliorating a disease, disorder, condition, or side effect or to decreasing the rate of advancement of a disease, disorder, condition, or side effect. The term also includes, within its scope, enhancing normal physiological function, palliative treatment, and partial remediation of a disease, disorder, condition, side effect, or symptom thereof.

As used herein, “treating” and “treatment” generally refer to obtaining a pharmacological and/or physiological effect. The effect may be prophylactic in terms of preventing or partially preventing a disease, symptom, or condition thereof.

As used herein, “hydrogel” refers to a three-dimensional polymer network to imbibe large amounts of water, which is used for the purpose of biomedical applications, including but not limited to the treatment of wound healing, cell culture, drug delivery, contact lenses, plastic surgery, and tissue regeneration. Hydrogels can chemically or physically contain various pharmaceutical drugs or immunomodulatory agents, including chemical drugs, proteins, peptides, nucleotides, and ions. Hydrogels also enable control of responses by modulation of the density and polarity of the polymer network, imparting the stimuli′ responsiveness to the polymer network

As used herein, “auxetics” refers to structures or materials that have a negative Poisson's ratio. When stretched, they become thicker perpendicular to the applied force. This occurs due to their particular internal structure and the way this deforms when the sample is uniaxially loaded. Auxetics can be single molecules, crystals, or a particular structure of macroscopic matter.

A “medical device” is any device intended to be used for medical purposes. For example, an instrument, apparatus, implement, machine, contrivance, implant, in vitro reagent, or other similar or related article, including a component part or accessory which is:
1. Recognized in the official National Formulary, or the United States Pharmacopoeia, or any supplement to them,
2. Intended for use in the diagnosis of disease or other conditions, or in the cure, mitigation, treatment or prevention of disease, in man or other animals or,
3. Intended to affect the struct or any function of the body of man or other animals, and which does not achieve its primary intended purposes through chemical action within or on the body of man or other animals
4, and which does not achieve its primary intended purposes through chemical action within or on the body of man or other animals and which is not dependent upon being metabolized for the achievement of its primary intended purposes.

An “implant” refers to a medical device manufactured to replace a missing biological structure, support a damaged biological structure, or enhance an existing biological structure. Medical implants are man-made devices, in contrast to a transplant, which is a transplanted biomedical tissue. Some implants can be bioactive, such as subcutaneous drug delivery devices in the form of implantable pills or drug-eluting stents. Examples of implants include, but are not limited to, sensory and neurological; cardiovascular; orthopedic; such as bone, joint, and tendon implants; contraceptive; cosmetic; and for any other organ or system.

By “biomaterial” is meant a substance that has been engineered to interact with biological systems for a medical purpose, either a therapeutic (treat, augment, repair, or replace a tissue function of the body) or a diagnostic one.

### General Description

The present disclosure relates to three-dimensional (3D) auxetics composed of, or comprising, a periodic network of 3D auxetic unit-cells which is fundamentally a cube form with a symmetrical arrangement of orthogonally patterned lines on each plane. This 3D auxetic can be configured for very high initial flexibility under compression resulting from the bending behavior of the lines in accordance with local rotations of joints where lines intersect. This 3D auxetic also can be configured with nonlinear stiffening behavior with negative Poisson's ratio, meaning that as compression is applied inducing line bending and eventual contact between vertical and horizontal parallel lines, compressive load will also increase at an increasing rate. Moreover, this 3D auxetic can be configured with a very high void fraction that can converge to approximately 92% as size increases, which can assure high permeability for high cell viability and functionality even at large sizes. In addition, the biological performance (for example, the ability to prevent inflammation, ability to accelerate tissue regeneration, etc.) of 3D auxetics can be improved by incorporating hydrogel-based biomaterials with or without cells, growth factors, or drugs.

### 3D Auxetic Unit-Cell

Example Set #1. 3D auxetic structures in the present disclosure are composed of a periodic network of 3D auxetic unit-cells. The 3D auxetic unit-cell is fundamentally a cube form with a symmetrical arrangement of orthogonally patterned lines having a square cross-section of 500×500 μm² on each plane, but not limited to this cross-sectional area. For example, the cross-sectional area can be 50×50 μm², 100×100 μm², 150×150 μm², 200×200 μm², 250×250 μm², 300×300 μm², 350×350 μm², 400×400 μm², 450×450 μm², 500×500 μm², 550×550 μm², 600×600 μm², 700×700 μm², 750×750 μm², 800×800 μm², 850×850 μm², 900×900 μm², 950×950 μm², or 1000×1000 μm², or any amount above, below, or in-between these values. Orthogonally patterned lines in the auxetic unit-cell can also have a circular cross-section of 50 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1000 μm, or any amount above, below, or in-between these values.

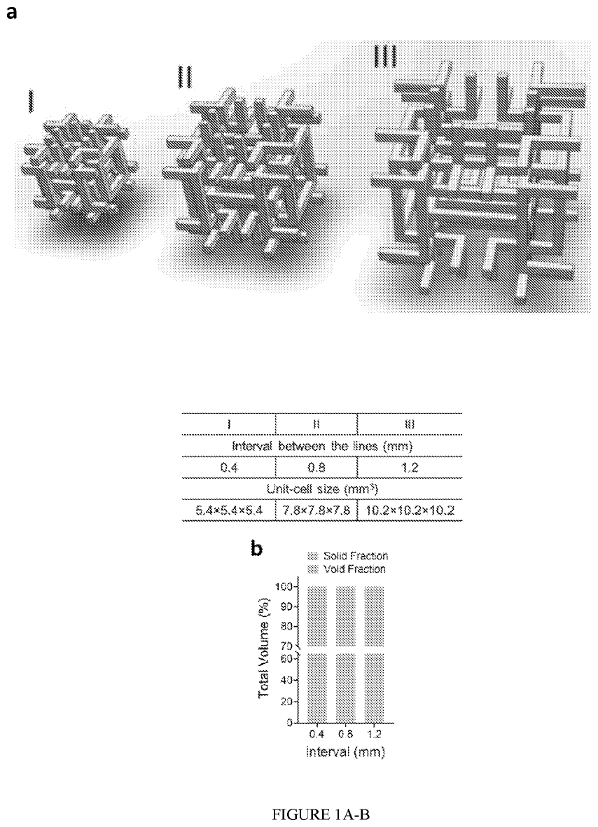

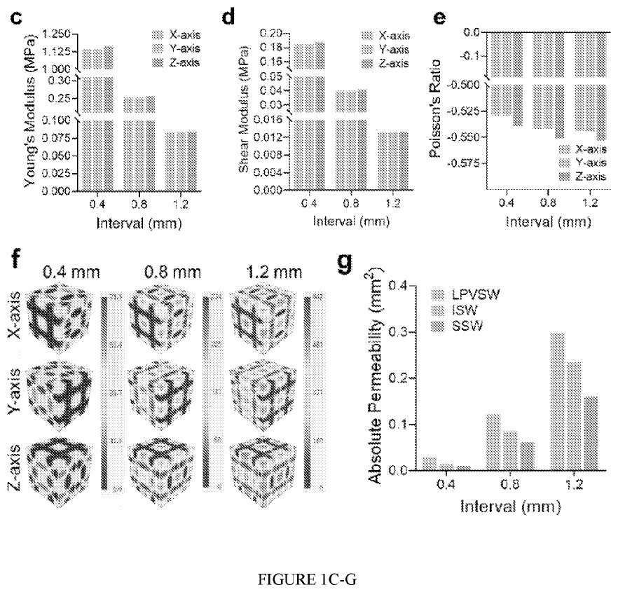

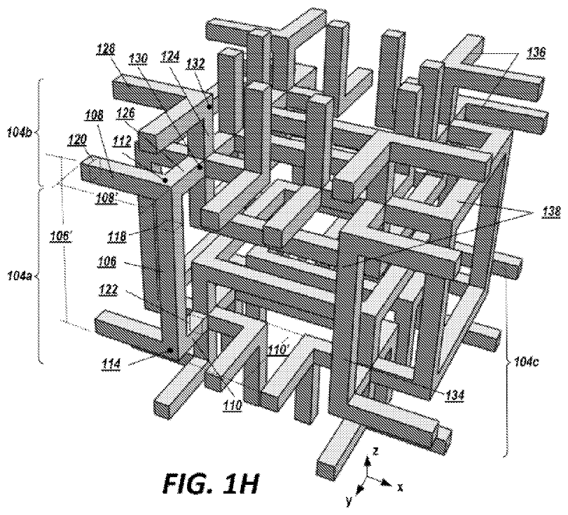

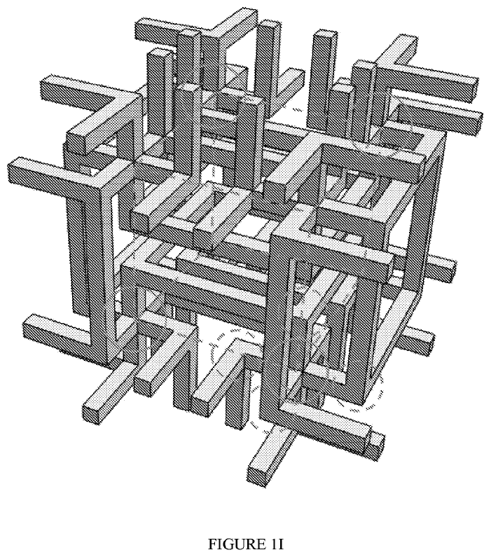

The lines symmetrically arranged on each plane intersect at the points where they bend at a 90° angle with the lines on surrounding perpendicular planes. Accordingly, the unit-cell can have eight main joints where the lines intersect at each vertex of a cubic form, as well as three minor joints around each main joint. This can be seen in FIG. 1.

Cubic size and void fraction of the auxetic unit-cell can increase as the interval between parallel lines increases. Effective mechanical properties of 3D auxetic unit-cell with different intervals were calculated through homogenization analysis to approximate the response to the external loading. A mixed uniform boundary condition was used for elasto-static simulation as it is particularly well adapted to isotropic and orthotropic materials. Interestingly, a three-times increase in the line interval (from 0.4 mm to 1.2 mm) can lead to a significant decrease (more than 10 times) in Young's and shear modulus, whereas there is a relatively slight change in Poisson's ratio. The orthotropic configuration of the auxetic unit-cell with symmetry properties was also confirmed with errors in the orthotropic approximation of 0.03719%, 0.07151%, and 0.1436% for intervals of 0.4 mm, 0.8 mm, and 1.2 mm, respectively. The absolute permeability of unit-cells calculated through viscous Strokes fluid flow simulation increases with increasing intervals. As the interval increases, the mean fluid velocity and consequential absolute permeability can significantly increase in all Stokes flow boundary conditions of linear pressure variation on side walls (LPVSW), impermeable side walls (ISW), and solid side walls (SSW).

The interval between parallel lines in the auxetic unit-cell can also be 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, 5.0 mm, or any amount above, below, or in-between these values.

In the example shown in FIG. 1H, the auxetic unit-cell is formed of a periodic network of orthogonally patterned regions 104, including a first orthogonally patterned region 104a and a second orthogonally patterned region 104b. The first orthogonally patterned region 104a is coupled to the second orthogonally patterned region 104b. Each of the first orthogonally patterned region 104a and the second orthogonally patterned region 104b includes a first longitudinal beam 106, a second longitudinal beam 108, and a third longitudinal beam 110. FIG. 1I is similar to FIG. 1H, and shows the structure of the unit-cube with the cube shape featured in the structure.

The first longitudinal beam 106 has a first length 106′. The second longitudinal beam 108 is fixably attached to the first longitudinal beam 106 at a first hinge region 112. The second longitudinal beam 108 has a second length 108′ and is orthogonally disposed with respect to the first longitudinal beam 106 in which the first length is different from the second length.

The third longitudinal beam 110 is fixably attached to the first longitudinal beam 106 at a second hinge region 114. The third longitudinal beam 110 has a third length 110′ and is orthogonally disposed with respect to the first longitudinal beam 106 and the second longitudinal beam 108 in which the third length 110′ is different from the first length 106′ and second length 108′.

The first orthogonally patterned region 104a and the second orthogonally patterned region 104b each forms a part of an auxetic unit-cell 101, having symmetrical arrangements of orthogonally patterned beams on each orthogonal plane.

The first hinge region 112, the first longitudinal beam 106, the second hinge region 114, and the third longitudinal beam 110 are configured to bend under load applied in a direction non-parallel to the longitudinal axis of the first longitudinal beam 106. In some embodiments, the first hinge region 112, the first longitudinal beam 106, the second hinge region 114, and the third longitudinal beam 110 are configured to bend under the load as a compression load from a bending behavior of the first longitudinal beam 106 and third longitudinal beam 110 when the first hinge region 112 and the second hinge region 114 bends under the same compression load.

In the example of FIG. 1, the first longitudinal beam 106 has a constant first cross-section area 118. The second longitudinal beam 108 has a constant second cross-section area 120. The third longitudinal beam 110 has a constant third cross-section area 122. In the example shown in FIG. 1H, the first cross-section area is the same as the second cross-section area, and the second cross-section area is the same as the third cross-section area. In other embodiments, the cross-section area between some connecting longitudinal beams can be different. The auxetic unit-cell structure is a low-density structure having a define-able void fraction between 60% and 95%. The high void fraction can provide high permeability for cell growth within the auxetic unit-cell.

Similarly, the second orthogonally patterned region 104b includes orthogonal positioned longitudinal beams. The example of FIG. 1H shows a fourth longitudinal beam 124, a fifth 126, and a sixth longitudinal beam 128. The fourth longitudinal beam 124 has a fourth length. The fifth longitudinal beam 126 is fixably attached to the fourth longitudinal beam 124 at a third hinge region 130. The fifth longitudinal beam 126 has a fifth length and is orthogonally disposed with respect to the fourth longitudinal beam 124. The fourth length is different from the fifth length. The sixth longitudinal beam 128 is fixably attached to the fourth longitudinal beam 124 at a fourth hinge region 132. The sixth longitudinal beam 128 has a sixth length and is orthogonally disposed with respect to the fourth longitudinal beam 124 and the fifth longitudinal beam 126, and the sixth length is different from the fourth length and fifth length.

The third orthogonally patterned region 104c is shown located proximal to the first orthogonally patterned region 104a such that the longitudinal beam 134 of the third orthogonally patterned region is parallel to the first longitudinal beam 106 of the first orthogonally patterned region 104a.

The orthogonally patterned regions include repeating L-shape structures 136 and repeating C-shape structures 138.

The longitudinal beams can have any cross-sectional area. In some embodiments, one or more of the longitudinal beams can have a constant cross-sectional area, which can be the same or different from that of other longitudinal beams. In some embodiments, one or more of the longitudinal beams can have a variable cross-sectional area, which can be the same or different geometry from that of other longitudinal beams. In some embodiments, some longitudinal beams have a constant area while others have a variable area. In some embodiments, different longitudinal beams of variable cross-sectional areas differ in their geometries. The longitudinal beams can have any cross-sectional geometry. This geometry can be rectangle, square, circle, triangle, hexagon, star, or any other shape. Different longitudinal beams can have the same or different cross-sectional geometries. The cross-sectional geometry can be constant throughout the length of a longitudinal beam or can be variable. The cross-sectional area and cross-sectional geometry of the longitudinal beams can depend on the intended end use of the apparatus, as well as the manufacturing method to be used. Different manufacturing methods can favor different cross-sectional areas and geometries. The cross-sectional area and geometry can also be impacted by any surface modification, for example a geometry with greater surface area can be desired should the apparatus comprise a bioactive surface coating. The longitudinal beams can be of any geometry. While the embodiment of FIG. 1H has longitudinal beams which are straight lines, alternative embodiments can have longitudinal beams which are curved.

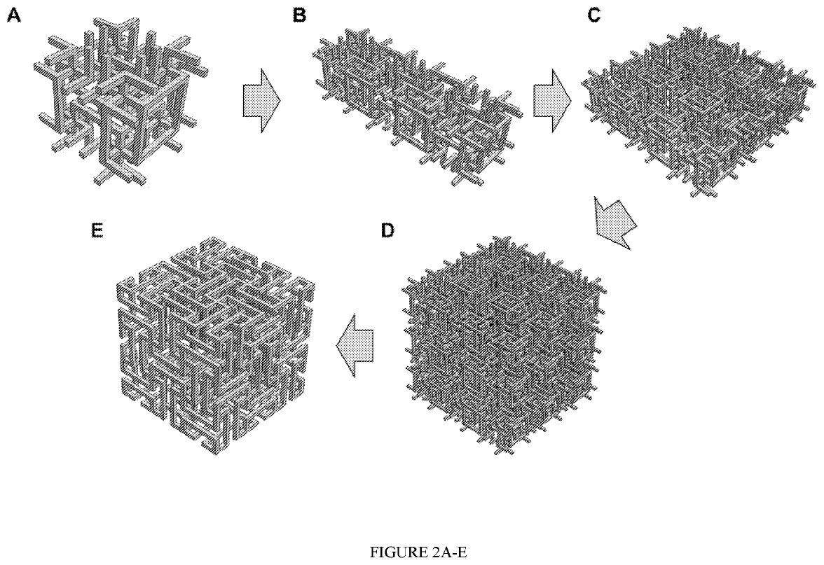

Example Set #2. In another set of examples, auxetic cube can be designed by assembling auxetic unit-cells and cutting protrusions on each plane. A 3×1 unit-cell array was created by placing the face of one unit-cell in contact with the face of other unit-cells (FIGS. 2A and B). Three of the 3×1 arrays were added in a same way to create 3×3 arrays (FIG. 2C). Three of the 3×3 arrays were added along Z-axis in a same way to create 3×3×3 arrays (FIG. 2D). Protrusions at the edge of an assembly were then cut to create auxetic cubes with all the lines connected, a flat exterior surface, and a specific size (FIG. 2E).

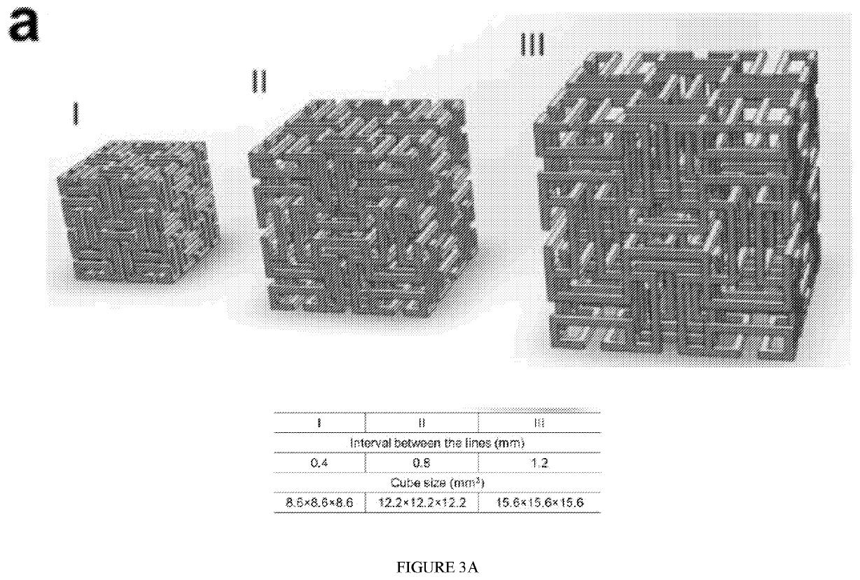

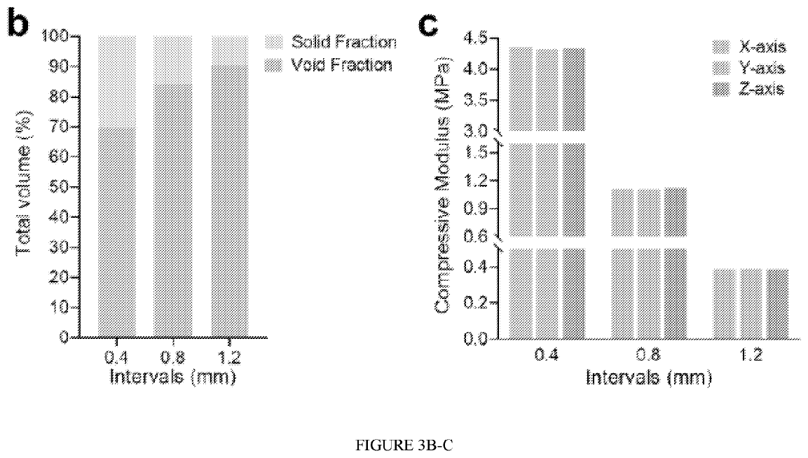

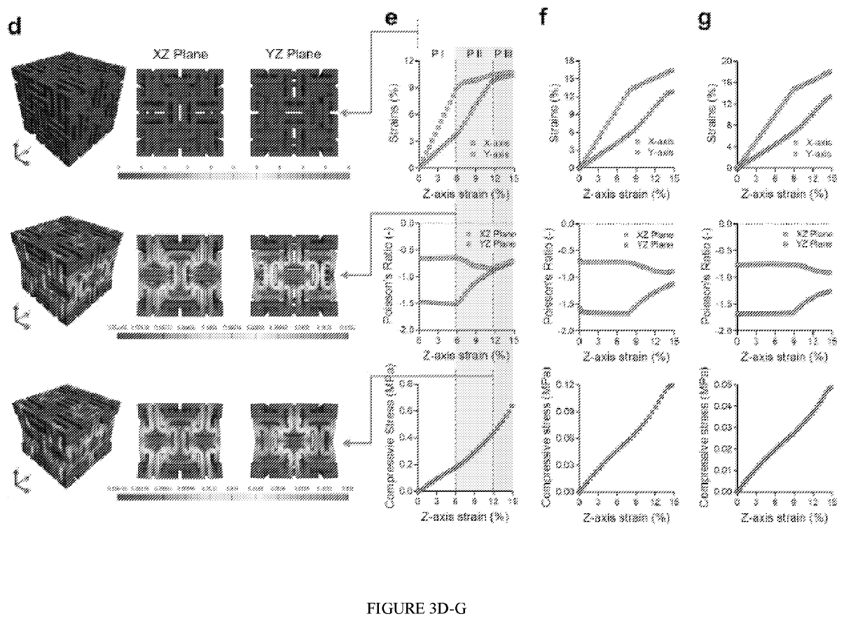

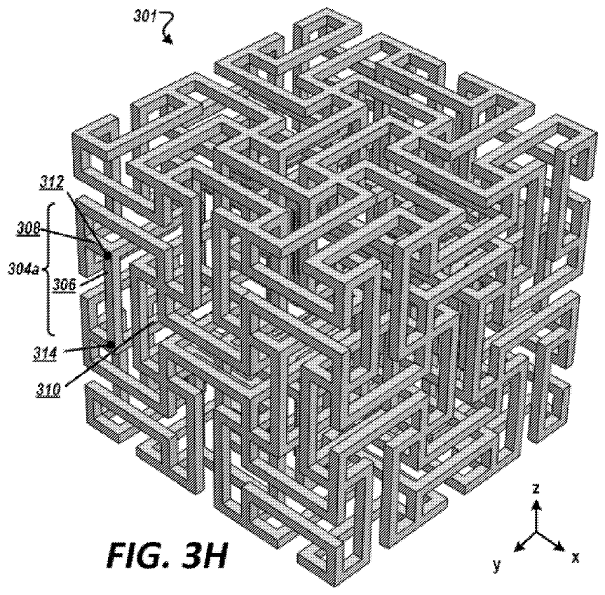

For example, auxetic cubes with three intervals can be designed through this process. (FIG. 3A-G). Auxetic cubes with a 1.2 mm interval can have a 90.394% of void fraction while it is 69.877% in a 0.4 mm interval. FE simulation was also performed using three auxetic cubes to analyze the mechanical properties and deformation behavior under uniaxial compression. Note that a general nonlinear large deformation analysis allowing contact between the lines of the auxetic cube was performed as opposed to the previous homogenization analysis performed, which was linear that did not account for contact.

The auxetic cubes shown in FIG. 3A have similar values of compressive modulus in all X-, Y-, and Z-directions. This mechanical property, regardless of compressive directions, unlike other 3D auxetics, assures the constant mechanical behavior under variable loading conditions, which can avoid the risk of unexpected weakness or failure in particular loading directions. Similar to elasto-static analysis results, compressive modulus can decrease more than 10 times with increasing intervals 3 times. The auxetic cube with the 1.2 mm interval has the smallest compressive modulus of 0.385 MPa, while it is 4.348 MPa in the case of a 0.4 mm of interval. As expected from those FE simulation results, the interval between the parallel lines is a fundamental design parameter that modulates the flexibility and permeability of the auxetic cube with the same line thickness.

All auxetic cubes with different intervals show similar non-linear deformation behavior under uniaxial compression, which can be explained in three structures according to the contact occurring between the parallel lines. In the case of the auxetic cube with a 0.4 mm interval, orthogonally patterned lines composing an auxetic cube are straight and macro pores between the lines are clearly visible before compression. As the compression is applied from the top, vertical and horizontal lines can start to bend by simultaneous rotation of the joints inside the cube. In the phase I, X- and Y-axis strains increase linearly, and the slope of the Y-axis strain is steeper than that of the X-axis. As a consequence of the linear increase of X- and Y-axis strains, the absolute value of Poisson's ratio (PR) on XZ and YZ planes can be quite consistent, and compressive stress also increases linearly while local stress concentration occurs at the joints and vertical lines in the midmost of the cube. The bending of vertical lines in the midmost of the YZ plane can be most severe, and they can contact each other first, whereas vertical lines on the XZ plane and horizontal lines on both planes are still apart at approximately 6% of Z-axis strain. In phase II, the absolute value of PR on YZ planes can decrease as the increase of the Y-axis strain becomes steady, whereas the Z-axis strain increases with a steeper slope. However, the X-axis strain increases with a steeper slope than phase I as the Z-axis strain increases, and the absolute value of PR on XZ planes consequentially can increase. Interestingly, the Poisson's ratio on the XZ and YZ planes eventually can converge to one value as the X-axis strain becomes the same as the Y-axis strain at approximately 13% of the Z-axis strain. All adjacent horizontal lines in the middle of the XZ and YZ planes can contact each other at this point. In phase III, the X- and Y-axis strains can become steady at a similar level, and PR on XZ and YZ planes consequently can converge to the same value as all horizontal and vertical lines in the midmost of the cube have contacted each other. An increase in compressive stress can be significantly accelerated in this structure. Although auxetic cubes with the 0.8 mm and 1.2 mm line intervals can show a similar non-linear strain stiffening behavior to the 0.4 mm line interval, the delayed contact between the lines due to large spacing can shift the stiffening to an increased Z-axis strain with significant decreases in compressive stress. As a consequence of the increase in the interval between the lines, the lines can have a higher slenderness ratio and greater space that precipitates buckling, induced by rotations of joints, before contacting each other. This large amount of buckling can lead to a significant increase in the X- and Y-axis strains in response to the Z-axis compression, which can result in more severe deformation. More importantly, initial high flexibility with stiffening under increased deformation, comparable to native soft tissues, can be achieved.

In the example shown in FIG. 3H, the auxetic cube employs an auxetic unit-cell formed of a periodic network of orthogonally patterned regions 304, including a first orthogonally patterned region 304a. The first orthogonally patterned region 304a is coupled to the second orthogonally patterned region. Each of the first orthogonally patterned region 304a includes a first longitudinal beam 306, a second longitudinal beam 308, and a third longitudinal beam 310.

The first longitudinal beam 306 has a first length. The second longitudinal beam 308 is fixably attached to the first longitudinal beam 306 at a first hinge region 312. The second longitudinal beam 308 has a second length and is orthogonally disposed with respect to the first longitudinal beam 306.

The third longitudinal beam 310 is fixably attached to the first longitudinal beam 306 at a second hinge region 314. The third longitudinal beam 310 has a third length and is orthogonally disposed with respect to the first longitudinal beam 306 and the second longitudinal beam 308 in which the third length is different from the first length and second length.

The first orthogonally patterned region 304a forms a part of an auxetic unit-cell 301, having symmetrical arrangements of orthogonally patterned beams on each orthogonal plane.

The orthogonally patterned regions include repeating L-shape structures and repeating C-shape structures.

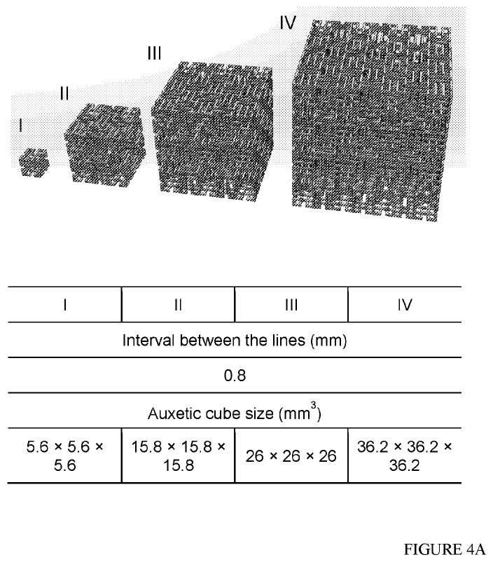

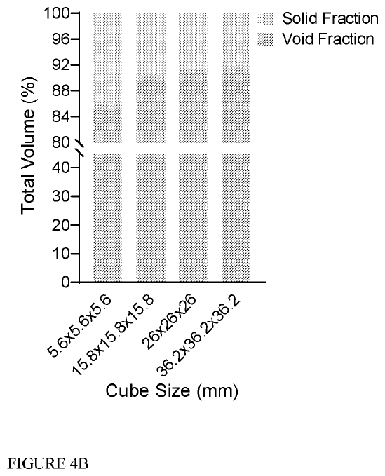

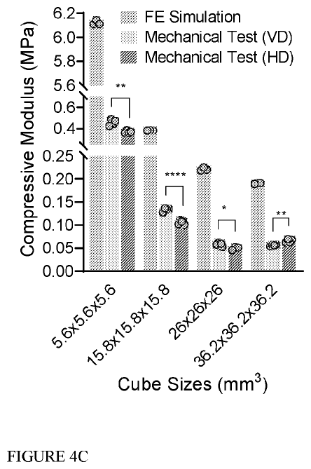

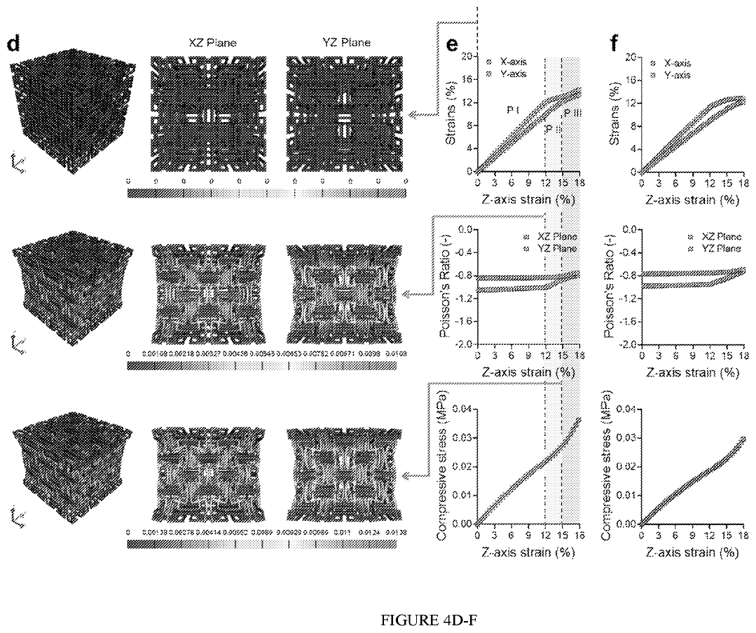

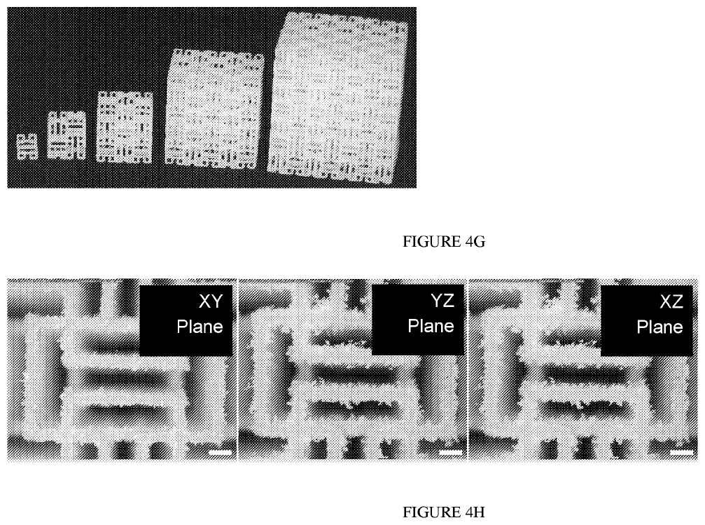

Mechanical Analysis. A study was conducted involving an in-depth analysis of the mechanical performance of auxetic cubes of different sizes. The study used both FEA and experimental approaches. In the study, the auxetic cube size was selected as being dependent on the number of unit-cells assembled from the same line thickness and the line interval. Given that the number of unit-cells can play a critical role in modulating the structural and mechanical properties of the auxetic cube, the study analyzed the effect of the size on mechanical and biological performance. In the study, auxetic cubes ranging from 5.6×5.6×5.6 mm to 36.2×36.2×36.2 mm were designed in 12 mm intervals (FIG. 4A). Contemplated herein, however, are auxetic cubes ranging in size from 1.0×1.0×1.0 mm, 1.5×1.5×1.5 mm, 2.0×2.0×2.0 mm, 2.5×2.5×2.5 mm, 3.0×3.0×3.0 mm, 3.5×3.5×3.5 mm, 4.0×4.0×4.0 mm, 4.5×4.5×4.5 mm, 5.0×5.0×5.0 mm, 5.5×5.5×5.5 mm, 6.0×6.0×6.0 mm, 6.5×6.5×6.5 mm, 7.0×7.0×7.0 mm, 7.5×7.5×7.5 mm, 8.0×8.0×8.0 mm, 8.5×8.5×8.5 mm, 9.0×9.0×9.0 mm, 9.5×9.5×9.5 mm, 10×10×10 mm, 11.0×11.0×11.0 mm, 11.5×11.5×11.5 mm, 12.0×12.0×12.0 mm, 12.5×12.5×12.5 mm, 13.0×13.0×13.0 mm, 13.5×13.5×13.5 mm, 14.0×14.0×14.0 mm, 14.5×14.5×14.5 mm, 15.0×15.0×15.0 mm, 15.5×15.5×15.5 mm, 16.0×16.0×16.0 mm, 16.5×16.5×16.5 mm, 17.0×17.0×17.0 mm, 17.5×17.5×17.5 mm, 18.0×18.0×18.0 mm, 18.5×18.5×18.5 mm, 19.0×19.0×19.0 mm, 19.5×19.5×19.5 mm, 20×20×20 mm, 21.0×21.0×21.0 mm, 21.5×21.5×21.5 mm, 22.0×22.0×22.0 mm, 22.5×22.5×22.5 mm, 23.0×23.0×23.0 mm, 23.5×23.5×23.5 mm, 24.0×24.0×24.0 mm, 24.5×24.5×24.5 mm, 25.0×25.0×25.0 mm, 25.5×25.5×25.5 mm, 26.0×26.0×26.0 mm, 26.5×26.5×26.5 mm, 27.0×27.0×27.0 mm, 27.5×27.5×27.5 mm, 28.0×28.0×28.0 mm, 28.5×28.5×28.5 mm, 29.0×29.0×29.0 mm, 29.5×29.5×29.5 mm, 30×30×30, 31.0×31.0×31.0 mm, 31.5×31.5×31.5 mm, 32.0×32.0×32.0 mm, 32.5×32.5×32.5 mm, 33.0×33.0×33.0 mm, 33.5×33.5×33.5 mm, 34.0×34.0×34.0 mm, 34.5×34.5×34.5 mm, 35.0×35.0×35.0 mm, 35.5×35.5×35.5 mm, 36.0×36.0×36.0 mm, 36.5×36.5×36.5 mm, 37.0×73.0×37.0 mm, 37.5×37.5×37.5 mm, 38.0×38.0×38.0 mm, 38.5×38.5×38.5 mm, 39.0×39.0×39.0 mm, 39.5×39.5×39.5 mm, 40×40×40 mm, or any value above, below or in-between these sizes.

The void fraction of the auxetic cube can increase with the larger size. The void fraction of the auxetic cube of 5.6×5.6×5.6 mm can be significantly lower than other sizes but, from the size of 15.8×15.8×15.8 mm upward, the void fraction can converge gradually to approximately 92%. Consequently, the compressive modulus of auxetic cubes calculated from FE simulation can converge to a lower bound with increasing cube size. The compressive modulus of 5.6×5.6×5.6 mm auxetic cube can be significantly higher than other sizes. In fact, this size of auxetic cube resists only by buckling of lines against the uniaxial compression as this size does not contain any valid joints, which are expected to rotate and induce the buckling of the lines to absorb the compressive force inside the structure. In other sizes of auxetic cube, the compressive modulus decreases with increasing cube size, converging to approximately 0.15 MPa.

Under uniaxial compression, all auxetic cubes or structures show similar non-linear stress-strain responses, but the deformation behavior of auxetic cubes with larger sizes than 15.8×15.8×15.8 mm is quite different from smaller sizes. In the case of the auxetic cube of 26×26×26 mm size, PR on XZ and YZ planes are almost constant with a much smaller difference between them in consequence of linear increases of X- and Y-axis strains with similar slopes in the phase I, which means that deformation behavior on both planes becomes similar. At approximately 12% of Z-axis strain, the horizontal parallel lines contact first in the middle of XZ plane followed by contact of vertical parallel lines in the middle of YZ plane. In addition, the absolute value of PR in phase II does not increase as the X-axis strain can increase with same slope as phase I while the Y-axis strain becomes steady. As a result, PR on both planes becomes quite similar. After all vertical and horizontal parallel lines in the middle of both planes contact at approximately 15% of Z-axis strain, PR on both planes converges to similar and compressive modulus start increasing with a steeper slope in phase III. Local stress concentration occurs at vertical lines in the same direction as the compression direction, as well as in the joints in the midmost of the cube under compression. The 36.2×36.2×36.2 mm auxetic cube structure shows a lower stress-strain response and similar deformation behavior to the 26×26×26 mm structure.

In some embodiments, the scaling up of auxetic unit-cell may be necessary to assure the reproducibility when fabricating the auxetic structure using the fabrication method with poor resolution. It is expected that scaling up of auxetic unit-cell with increased line widths and intervals brings a corresponding change in the mechanical properties and behavior of the auxetic structure. Determination of appropriate line interval of auxetic unit-cell for a certain line width is important to maintain high flexibility of the scaled up auxetic structure. To this end, scale up design of the auxetic unit-cell was performed by increasing the line width to 1 mm and line intervals to 2.4, 3.0 and 3.5 mm. Auxetic cubes that have same number of the unit-cells were then designed and the proper interval between the parallel lines in the scaled up auxetic unit-cell was determined through FE simulations by comparing the load and displacement curves of the scaled up auxetic cubes under the compression to the previous auxetic cube of 0.5 mm line width and 1.2 mm line interval. Under the uniaxial compression, the initial slope in the load and displacement curve of the scaled up auxetic cube of 1 mm line width and 3.5 mm line interval was slightly lower than that of the auxetic cube of 0.5 mm line width and 1.2 mm line interval. Interestingly, this result led significant decrease (more than around 3-fold) in the compressive modulus of the scaled up auxetic cube as a result of increase in the cross-sectional area by increasing line width and interval. The compressive modulus of the auxetic cube with 1 mm line width and 3.5 mm line interval was 0.107 MPa while that of the auxetic cube with 0.5 mm line width and 1.2 mm line interval was 0.359 MPa. It was concluded that the initial flexibility of the auxetic cube significantly enhanced by scaling up design. As discussed, in some embodiments, the auxetic structure described herein can be manufactured using additive manufacturing techniques, for example, being 3D printed. Each structure can be specifically designed with its use in mind, since additive manufacturing can allow for rapid and economical small-scale manufacturing. For example, medical imaging can be used to design a patient-specific implant that conforms to a patient's anatomical 3D boundaries. These images and/or parameters could be obtained from one or more imaging systems such as computed tomography (CT), CT-fluoroscopy, fluoroscopy, magnetic resonance imaging (MRI), ultrasound, positron emission tomography (PET) and X-Ray systems or any other suitable imaging systems. The medical image data and/or parameters received from the imaging system could provide a two-dimensional (2D), three-dimensional (3D) or four-dimensional (4D) model of an anatomical structure, system or region of the patient. Visualization and/or analysis of these images could be conducted using software, for example Mimics by Materialise, Simpleware ScanIP by Synopsys, IDL, and Amira. The image-based design of the apparatus may be created using computer-aided-design (CAD) software, for example MATLAB, Mathematica, or any other design programs known in the art. The image used could have been captured at any time and may have had an unrelated initial purpose.

The auxetic structure can be fabricated using additive manufacturing methods including, but not limited to, selective laser sintering (SLS), extrusion, fused deposition modeling (FDM), fused filament fabrication (FFF), PolyJet, stereolithography (SLA), digital light processing (DLP), multi jet fusion (MJF), electron beam melting (EBM), direct metal laser sintering (DMLS), any combination thereof, and any other additive manufacturing technique existing or to be developed in the future. Before and/or after printing, additional post processing steps may be used, for example cleaning, thermal treatment, plasma treatment, and exposure to UV light. The apparatus may be comprised of multiple components, for example a polymer support structure with a hydrogel in the voids. Components may be manufactured out of the same or different materials and using the same or different manufacturing methods. Assembly of multiple components may be required.

In the present disclosure, selective laser sintering-based 3D printing method was used to fabricate auxetic cubes of 1.2 mm line interval with different sizes from PCL (FIG. 4C). Orthogonally patterned lines with a rough surface caused by powder particles aggregation are confirmed in magnified images. It is also shown that the line width from the XY plane is thinner than that from the YZ and XZ planes. This difference in line width is associated with the inherent printing characteristics of SLS, specifically the directional dependence of sintering density, caused by accumulated exposure to the laser during the printing process. Interestingly, the printed auxetic cubes have a ⅓ lower compressive moduli than those in the FE simulation results. Although the compressive modulus measured from the printed auxetic cubes was slightly different according to the compression direction (vertical and horizontal), it shows a similar trend of decreasing with increasing cube sizes, and converges to approximately 0.05 MPa. Through this analysis, stable scalability of the auxetic cube printed by SLS was confirmed with converged mechanical properties and behaviors.

Fundamentally, although the mechanical properties and behavior of the 3D structures depend on the inherent properties of the material used, they can also be tuned significantly through internal architecture design. As disclosed herein, PCL was used as a printing material for auxetic cubes with the ultimate goal of clinical translation. Although PCL has been widely applied to clinical translation, its application is limited to stiff medical devices or hard tissue reconstruction scaffolds due to the inherent relatively stiff and linear mechanical properties of PCL. Given that PCL has good printability as well as clinically proven biocompatibility, expanding the applicability of 3D printing of PCL is highly desirable to develop clinically viable substrates for the reconstruction of large-volume soft tissue defects. In this regard, a novel architected auxetic design was developed to create PCL 3D structures for soft tissue engineering and validated its high flexibility and non-linear mechanical behavior comparable to soft tissues.

Selective laser sintering (SLS) based 3D printing of Poly-&-caprolactone (PCL) was employed to create 3D auxetic cubes with the ultimate goal of clinical translation. SLS is one of the 3D printing modalities which has been used to produce medical devices and tissue reconstruction scaffolds for clinical translation. Compared to other 3D printing modalities, SLS has high design flexibility that allows intricate and complex geometries as support is provided by unsintered powder surrounding and holding the printing structure during the printing process. Rapid mass manufacturing achieved by high-speed laser scanning is also one of the greatest advantages of SLS. For these reasons, SLS is a preferred method for creating auxetic architectures in large volumes with complex anatomic geometries.

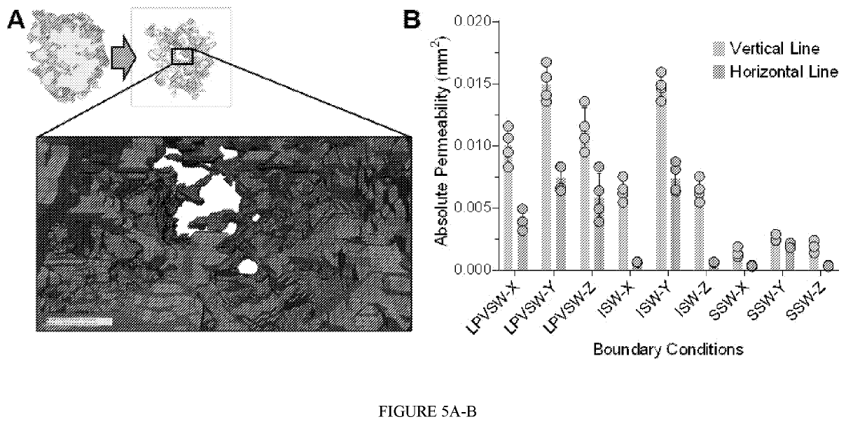

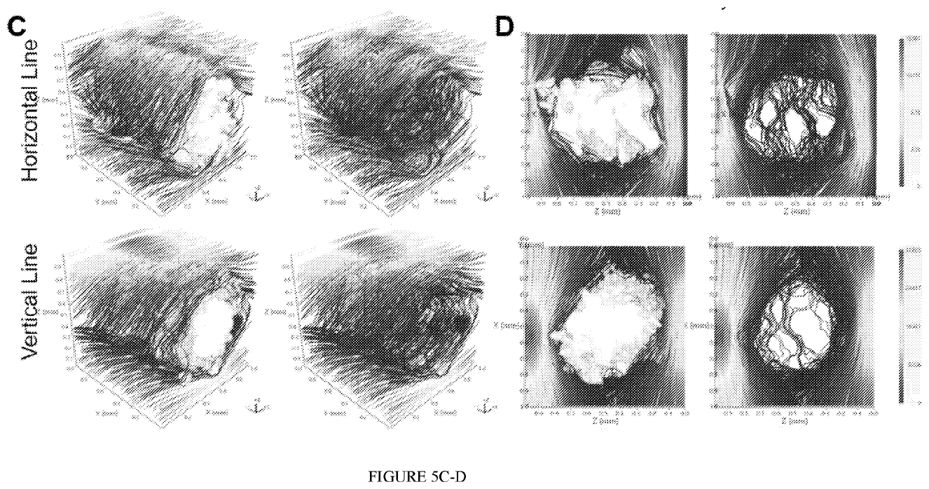

Another important characteristic of SLS is that it allows for the inclusion of microscale voids within the designed structure through powder particle aggregation. This internal microporosity can further enhance flexibility, permeability, and nonlinear elastic compliance of the auxetics, all desirable features in large volume soft tissue engineering. As expected, auxetic cube lines include microscale voids that can deviate from the solid design (FIG. 5G). The difference in the void fraction of segmented vertical and horizontal, single lines is also associated with the directional dependence of sintering density. This microporosity can increase the flexibility of individual lines within the auxetic cubes and eventually enhances the effect of the auxetic design on flexibility and compliance. The exceptional flexibility and non-linear mechanical behavior of auxetic cubes, achieved despite the relatively stiff and linear mechanical properties of PCL, is a consequence of the synergy effect of the inherent printing characteristic of SLS and the architected auxetic design. The microporosity effect on the permeability is also confirmed numerically through a viscous Strokes fluid flow simulation using a reverse volume image (1×1×1 mm) constructed from high-resolution uCT scanning of segmented auxetic cube single lines. Streamlines of the fluid flow infiltrating through the microscale voids within the single lines are observed, and the difference in the amount of infiltrating streamlines (more abundant in horizontal lines than vertical lines) is congruent with the differences in void volumes. Absolute permeability, calculated from the average velocity of streamlines flowing through reverse volume, including microscale voids, is higher in vertical lines than horizontal lines in all boundary conditions, as the infiltrated streamlines through the voids inside the line can disturb the fluid flow across the reverse volume.

Regarding voids, the void or voids within each base unit can have any suitable shape and configuration. The base shape of the void is preferably selected to provide desired tension and compression properties to implant. In some embodiments, wherein the base geometric shape of the voids comprises a spherical shape or at least one regular non-spherical shape such as ovoid, ellipsoid (including rugby ball shaped), cubic, cuboid, parallelepiped, hyperboloid, conical, octahedron, or other regular 3D polygon shapes. In preferred forms, the void comprises a spherical, ovoid, or ellipsoid, more preferably spherical, or ovoid, and yet more preferably spherical.

In other embodiments, the void or voids can have a non-regular shape. For example, in some embodiments, the void or voids can be formed from a combination of interconnected void shapes such as ovoid, ellipsoid (including rugby ball shaped), cubic, cuboid, parallelepiped, hyperboloid, conical, octahedron, or other regular 3D polygon shapes.

In yet other embodiments, the base geometric shape of the voids comprises an optimized shape, thus comprising an optimized shape void. It is to be understood that an optimized shaped void is a shaped void having a configuration and shape derived from optimization algorithms, preferably bi-directional evolutionary structural optimization (BESO), to provide the desired response properties. The void shape therefore has an optimized shape to provide these responses. Such optimized shaped voids typically have complex shapes and can comprise an amalgamation of a number of different regular shapes. Furthermore, optimized-shaped voids can comprise two or more separate void shapes within the base unit. For example, a base unit may include three separate void spaces, the void spaces being generally located at the sides and one void around the geometric center of the base unit. Preferably, the void is shaped to assist in providing the auxetic structure with at least one of a negative linear compression (NLC), negative area compression (NAC), zero linear compression (ZLC), or zero area compression (ZAC) behavior when under pressure.

The base unit material can be any suitable base material. In some embodiments, the base unit material comprises a polymeric material. Exemplary polymeric materials include at least one of an unfilled or filled vulcanized rubber, natural or synthetic rubber, crosslinked elastomer, thermoplastic vulcanizate, thermoplastic elastomer, block copolymer, segmented copolymer, crosslinked polymer thermoplastic polymer, filled or unfilled polymer or epoxy. In other embodiments, base unit material comprises metallic and ceramic, and composite materials. Exemplary metals include aluminum, magnesium, titanium, iron, and alloys thereof.

The apparatus can comprise one or more materials. In some embodiments, the base unit material comprises a biocompatible material, preferably a biocompatible polymeric material to ensure the safety and efficacy of the apparatus when implanted. The material may also in some examples be a biodegradable (also referred to as “bioabsorbable” and “bioresorbable”) material. This may allow for the material to resorb into the body over time as tissue grows around and/or through the apparatus. Biodegradable polymers which may be used include but are not limited to polycaprolactone (PCL), polysebacic acid, poly(octaindiolcitrate), polydioxanone, polygluconate, poly(lactic acid) polyethylene oxide copolymer, modified cellulose, polyhydroxybutyrate, polyamino acids, polyphosphate ester, polyvalerolactone, poly-6-decalactone, polylactonic acid, polyglycolic acid, polylactides, polyglycolides, copolymers of the polylactides and polyglycolides, polyhydroxybutyric acid, polyhydroxybutyrates, polyhydroxyvalerates, polyhydroxybutyrate-co-valerate, poly(1,4-dioxane-2,3 one), poly(1,3-dioxane-2-one), poly-para-dioxanone, polyanhydrides, polymaleic acid anhydrides, polyhydroxy methacrylates, fibrin, polycyanoacrylate, polycaprolactone dimethylacrylates, poly-3-maleic acid, polycaprolactone butyl acrylates, multiblock polymers from oligocaprolactonediols and oligodioxanonediols, polyether ester multiblock polymers from PEG and poly(butylene terephthalates), polypivotolactones, polyglycolic acid trimethyl carbonates, polycaprolactone glycolides, poly(methyl glutamate), poly(DTH-iminocarbonate), poly(DTE-co-DT-carbonate), poly(bisphenol A-iminocarbonate), polyorthoesters, polyglycolic acid trimethyl carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly [p-(carboxyphenoxy) propane], polyhydroxy pentanoic acid, polyanhydrides, polyethylene oxide propylene oxide, and combinations thereof.

In some embodiments, the apparatus comprises a non-biodegradable biocompatible polymer. These polymers do not substantially resorb, dissolve or otherwise degrade after implantation in a patient, under typical physiological conditions. Suitable biomedically acceptable non-biodegradable biocompatible polymers include but are not limited to polyaryl ether ketone (PAEK) polymers (such as polyetherketoneketone (PEKK), polyetheretherketone (PEEK), and polyetherketoneetherketoneketone (PEKEKK)), polyolefins (such as ultra-high molecular weight polyethylene, which may be crosslinked, and fluorinated polyolefins such as polytetrafluorethylene (PTFE) or high density porous polyethylene), polyesters, polyimides, polyamides, polyacrylates (such as polymethylmethacrylate (PMMA)), polyketones, polyetherimide, polysulfone, polyurethanes, and polyphenolsulfones. The apparatus may comprise multiple biocompatible polymers, including one or more biodegradable biocompatible polymers, one or more non-biodegradable biocompatible polymers, and any combinations thereof. Thus, in certain variations, a portion of the apparatus may be biodegradable, while another portion is permanent and non-biodegradable.

The structure and configuration of the auxetic material of the present invention can be determined using a number of methods. In some embodiments of the present invention, the configuration of a structured porous material according to the present invention is determined using structural optimization algorithms, such as bi-directional evolutionary structural optimization (BESO) modeling techniques. The apparatus can further comprise one or more bioactive materials. More specifically, the first biocompatible polymeric material and the second biocompatible polymeric material may independently comprise a bioactive material. Depending on such factors as the bioactive material, the structure of the apparatus, and its intended use, the bioactive material may be coated on a surface of the tissue scaffold component, or otherwise infused together with hydrogel-based biomaterials in the pores or voids of the structure, or mixed or compounded within the biocompatible polymeric material of the apparatus.

Bioactive materials can include any natural, recombinant or synthetic compound or composition that provides a local or systemic therapeutic benefit. In various embodiments, the bioactive material promotes healing and growth of tissue. Bioactive materials among those useful herein include cell adhesion factors, isolated tissue materials, growth factors, peptides and other cytokines and hormones, pharmaceutical actives, nanoparticles, and combinations thereof. Cell adhesion factors include, for example, the RGD (Arg-Gly-Asp) sequence or the IKVAV (Ile-Lys-Val-Ala-Val) sequence. Growth factors and cytokines useful herein include erythropoietin (EPO), transforming growth factor-beta (TGF-β), including the five different subtypes (TGF-β 1-5); bone morphogenetic factors (BMPs, such as BMP-2, BMP-2a, BMP-4, BMP-5, BMP-6, BMP-7 and BMP-8); platelet-derived growth factors (PDGFs); insulin-like growth factors (e.g., IGF I and II); fibroblast growth factors (FGFs), vascular endothelial growth factor (VEGF), epidermal growth factor (EGF) and combinations thereof. Examples of pharmaceutical actives include antimicrobials, antifungals, chemotherapeutic agents, and anti-inflammatoires. Examples of antimicrobials include triclosan, sulfonamides, furans, macrolides, quinolones, tetracyclines, vancomycin, cephalosporins, rifampins, aminoglycosides (such as tobramycin and gentamicin), and mixtures thereof.

In certain embodiments, the apparatus comprises a bioactive material in the form of a biomaterial that may be selected from the group consisting of: an isolated tissue material, a hydrogel, acellularized dermis, an acellularized tissue matrix, a composite of acellularized dermis matrix and designed polymer, or a composite of acellularized tissue matrix and designed polymer, and combinations thereof. An isolated tissue material may include an autologous or allogeneic tissue sample (such as an explant harvested in the patient by a punch biopsy to provide tissue sample). In other aspects, an isolated tissue material may include isolated or cultured cells (such as chondrocyte cells, hemopoietic stem cells, mesenchymal stem cells, such as adipose-derived mesenchymal stem cells, endothelial progenitor cells, fibroblasts, reticulacytes, and endothelial cells), whole blood and blood fractions (such as red blood cells, white blood cells, platelet-rich plasma, and platelet-poor plasma), collagen, fibrin, acellularized dermis, and the like. In one embodiment, the isolated tissue biomaterial may comprise a combination of porcine adipose-derived stem cells and/or bone marrow derived or induced pluoripotent stem cells with chondrocytes, which may be combined at ratios of about 1:1 to 10:1. Hydrogels are materials formed from lightly-crosslinked networks of natural or synthetic polymers, such as saccharides, which have high water contents such as 90% weight per volume or greater. Hydrogel crosslinking can be achieved by various methods including ionic, covalent chemical, or UV-initiated chemical crosslinking. Hydrogels used in the present disclosure are preferably biocompatible. Hydrogels may be formed from hyaluronic acid/hyaluronan, collagen, sodium alginate, gelatin-methacrylate (gelMA), polyethylene glycol (PEG), polyethylene glycol diacrylate (PEGDA), 2-hydroxyethyl methacrylate (HEMA)/poly(2-hydroxyethyl methacrylate) (pHEMA), polymethyl methacrylate (PMMA), polyacrylic acid, chitosan, poly(amino acids), poly(N-isopropylacrylamide) (PNIPAM), collagen, gelatin, fibronectin, chondroitin sulfate, surfactant gels (having greater than about 20% weight per volume poloxamers (e.g., commercially available as PLURONIC™ and BRU™), polydimethylsiloxane (PDMS) or dimethicone, epoxy, polyurethane, and the like. In one embodiment, a suitable hydrogel-based biomaterial may comprise hyaluronic acid and Type I collagen. In certain embodiments, the apparatus may have a biomaterial disposed on one or more surfaces that will contact tissue in the patient upon implantation. In some embodiments, hydrogel-based biomaterials such as collagen, alginate, gelatin-methacrylate (gelMA), and decellularized extracellular matrix (ECM) can be incorporated with or without cells or growth factors and drugs to infill the voids, intervals between the lines, of auxetic in the manufacturing process. The specific dose of medical drugs including antibiotics or other high-bioactivity materials like hydroxyapatite (HA) also can be incorporated in the hydrogel-based biomaterials. Accordingly, the biological performance of the auxetic structure can be improved.

In certain embodiments, the biocompatible polymeric material comprises a biodegradable polymer. In certain embodiments, the biocompatible and biodegradable polymeric material of the apparatus comprises polycaprolactone.

In certain embodiments, the biocompatible polymeric material further comprises a bioactive agent which may be selected from the group consisting of: be a cell adhesion factor, a growth factor, a peptide, a cytokine, a hormone, a pharmaceutical active, and combinations thereof.

In certain aspects, the biocompatible polymeric material further comprises a biomaterial. The biomaterials may be selected from the group consisting of: an isolated tissue material, a hydrogel, acellularized dermis, an acellularized tissue matrix, a composite of acellularized dermis matrix and designed polymer, or a composite of acellularized tissue matrix and designed polymer, and combinations thereof.

In certain aspects, the biocompatible polymeric material comprises a material selected from the group consisting of: nanoparticles, growth factors, cells, tissue infusions, and combinations thereof.

The auxetic material described herein can be configured as a scaffold for tissue or organ implant. Some embodiments, for example, may be configured for breast or facial reconstruction, or for calcaneal fat pad implants. The auxetic structure makes the present apparatus uniquely able to mimic mechanical properties of fat tissue and thus has an advantage in applications where the implant is intended to replace and/or regenerate adipose tissue, for example as breast implants. The ability for the apparatus to be of a patient-specific design further optimizes its ability to serve as such a breast or facial implant, since the patient can give input into their preferences as it concerns their physical appearance.

The global breast reconstruction market size is large and fast growing. The growth can be accredited to the increasing number of breast cancer patients globally. The increasing awareness about breast reconstruction procedures, technological advancement, and the availability of favorable reimbursement policies are also expected to impel the market growth. A large portion of women prefer breast reconstruction after mastectomy, making breast reconstruction an important application for this technology. Because of the reduced surgical duration and lack of donor sites, patients usually prefer implants in the breast reconstructing process. Most of the breast implant market currently is in silicone and saline devices. However, breast implant-based procedure still incurs short-term risk of infection and long-term risks of capsular contracture and implant rupture. These risks emphasize the need for a new tissue engineering strategy for breast reconstruction. PCL auxetic cube created by SLS based 3D printing in this invention successfully demonstrated initial high flexibility with non-linear stiffening behavior under large compressive deformation, all desirable features for soft tissue engineering including adipose tissue regeneration. Moreover, enhanced permeability achieved by bimodal pore distribution including designed macro pores as well as micro pores within the lines overcame the limited oxygen diffusion and nutrient transfer of gelMA hydrogel. This means that PCL auxetic structure can serve as an implant for large volume soft tissue regeneration including breast reconstruction.

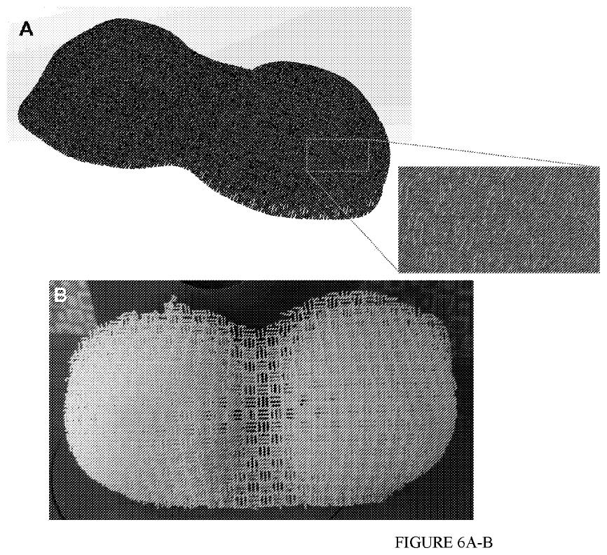

FIGS. 6A-6B is an embodiment of the apparatus configured as a patient-specific breast implant, designed to the contours of the patient's native breast anatomy. FIG. 6A is a computer aid design (CAD) model of the apparatus design, and FIG. 6B is a photograph of the apparatus after being 3D printed using an SLS method (specifically, using an EOS Formiga P110 printer) composed of PCL (specifically, sourced from Polysciences Inc.).

The apparatus described herein can also be configured as an in vitro tissue model. For example, the in vitro tissue model can comprise cardiac cells. Embodiments of the apparatus configured as an in vitro tissue model may comprise, but are not limited to, one or more of: cardiac myocytes, adipose cells, adipose derived stem cells, hepatocytes, human skeletal muscle cells, and liver cells. The apparatus, configured as in vitro cardiac, adipose, liver or other tissue models, may be utilized for any number of applications, for example, drug screening. Companies seeking to develop drugs and test their safety and efficacy can utilize these models to reduce the time and risk involved with alternative methods such as in vivo animal models and clinical trials. The apparatus can comprise a framework for a hydrogel, which has multiple applications in bioengineering.

In vitro diagnostics (IVD) are tests done on artificial models composed of cells, tissues, or other biological components relevant to organs of interest. IVD can be used for biological assessment, detecting and modeling of diseases and disorders, and drug discovery and screening. IVD can be also used in precision medicine to monitor a person's overall health to help cure, treat, or prevent diseases. The global need for IVD is large and continuing to grow, driven largely by the rise in incidences of chronic and infectious diseases, including but not limited to tuberculosis (TB), cancer, cardiovascular diseases, and diabetes. In addition, there is a substantial rise in the number of patients suffering from infectious diseases, such as gastrointestinal, respiratory, and sexually transmitted diseases (STDs). The increase in these diseases is expected to increase the demand for diagnostic devices, which drives the IVD market. The North American IVD market is primarily in applications concerning diabetes, cardiology, nephrology, infectious disease, oncology, drug testing, and autoimmune diseases.

In vitro cardiac tissue models, which can be created using the apparatus disclosed herein, can be used for diagnostics of cardiovascular disease, which is the leading cause of death worldwide. It can be also used for the detection of cardiac toxicity, which is a major hurdle for drug discovery and development, with high rates of post-approval withdrawal of medicines. Current drug approval methodology often concludes failure of drug development due to the high risks of in vivo interventions in clinical trials and impossibility to fully recapitulate human physiology in animal models. Human in vitro models of cardiac tissue that fully recapitulate human cardiac physiology and drug response are therefore required to understand and develop new drugs and strategies for treating cardiac diseases. 2D in vitro systems cannot accurately recapitulate the complex physiological conditions in the human heart due to their inability to mimic the dynamics of the biological and mechanical properties of the in vivo microenvironment. An in vitro cardiac tissue model should therefore be able to recapitulate the physiological conditions of the human heart, including 3D anisotropic movement, the orientation of the extracellular network, and circulation. The auxetic structures of the present invention can serve as a framework to create auxetic-based 3D cardiac tissue models. Some embodiments of these models can comprise hydrogel-based biomaterials incapsulating human induced pluripotent stem cell-derived cardiomyocytes (iCMs) or other cell sources with relevant bioactive factors. The negative Poisson's ratio behavior and high resilience and durability of the auxetic structures disclosed herein enable the creation of a novel 3D cardiac tissue model that can exhibit heartbeat-mimetic movement (i.e. repetitive contraction and expansion) under cyclic compressive loading.

Tissue engineering has been defined as an interdisciplinary field that applies the principles of engineering and life sciences toward the development of biological substitutes that restore, maintain, or improve tissue function. Three general strategies are employed in tissue engineering: use of isolated cells or cell substitutes, use of tissue-inducing substances, and use of cells placed on or within matrices.

In some embodiments, the auxetic biomaterials are used in the field of tissue engineering; non-limiting examples of tissue engineering may include auxetic cardiac patches and blood vessel repair. In other embodiments, the biomaterials are used for healing wounds, including but not limited to medical sutures. Other uses contemplated herein are those that require scaffolds emulating auxetic tissues. In some embodiments, the auxetic biomaterials are fabricated from traditional polyethylene glycol (PEG).

Cells are often implanted or ‘seeded’ into an artificial structure capable of supporting three-dimensional tissue formation. These structures, typically called “scaffolds”, are often critical, both ex vivo as well as in vivo, to allow cells to influence their own microenvironments. Scaffolds serve one or more of the following purposes: allow cell attachment and migration; deliver and retain cells and biochemical factors; enable diffusion of vital cell nutrients and expressed products; and exert certain mechanical and biological influences to modify the behavior of the cell structure.

Auxetic structure can also be used as an implant or for other biomedical uses. Examples include, but are not limited to, intervertebral discs, pedicle screws or other types of bone fixation elements, stents, shunts, hip implants, knee implants, cardiac patches, nasopharyngeal swabs, orthopedic surgical braces, and meshes for tissue and muscle implants.

The auxetic material described herein can also be used in sports protection, such as in helmets and pads or in personal protective devices used by military and law enforcement. Other examples of items that can be made using the auxetic materials can be found in Lvov et al. (Lvov VA, Senatov FS, Veveris AA, Skrybykina VA, Díaz Lantada A. Auxetic Metamaterials for Biomedical Devices: Current Situation, Main Challenges, and Research Trends. Materials (Basel). 2022 Feb. 15; 15 (4): 1439), herein incorporated in its entirety for its teaching concerning uses of auxetic materials.

An advantage of this new 3D auxetic design and structure compared to previous disclosed auxetic structures is 1) higher flexibility and nonlinear behavior than previous structures, 2) cubic symmetry to provide sufficient load-bearing in multiple directions compared to previously disclosed structures and 3) large permeability due to a novel bimodal, two-scale porosity and at hundreds of microns and 1-10 microns.

One of the fabrication methods of the auxetic structure is selective laser sintering (SLS) based 3D printing that is currently used to fabricate airway splint devices for clinical application. Therefore, this auxetic structure fabrication method is safe for implantable devices. Furthermore, This 3D printing method allows for the shape and size of the 3D auxetic structure to be customized according to the patient's defect as well as for the mechanical properties of the 3D auxetic structure to be customized according to the target tissue by controlling line width and intervals between the lines.

Moreover, the fabrication method of the 3D auxetic structure in this disclosure can include the use of various biomaterials with or without drugs or other biologic materials, as well as hydrogel-based biomaterials with or without cells or growth factors. Accordingly, this fabrication method can produce the 3D auxetic structure with customized biologic performance for specific target tissue by incorporating proper material combinations.

A number of 2D or 3D architected auxetic designs, including re-entrant Honeycomb, have been developed and disclosed for various applications. However, the 3D auxetic unit-cell in the present embodiment includes more joints where lines intersect than in previous designs, resulting from the symmetrical arrangement of orthogonally patterned lines. Under compression, these joints rotate and induce bending of the lines between the joints. This bending facilitates absorption of the compressive load resulting in high initial flexibility. This 3D auxetic unit-cell also has an orthotropic configuration with symmetric mechanical properties that are similar along the X-, Y-, and Z-axis. In contrast to other existing 3D auxetics, this cubic symmetric mechanical property ensures the same stiffness along all three axes to resist variable loading conditions from different directions, which can avoid the risk of unexpected weakness or failure in a particular loading direction.

The aforementioned additive manufacturing fabrication method allows the creation of 3D auxetic structures with different line widths, intervals between lines, and sizes. Accordingly, the mechanical properties and behavior of 3D auxetic structures can be customized as needed. Furthermore, when creating the 3D auxetic structure using selective laser sintering (SLS) based 3D printing, the auxetic structure has a bimodal pore distribution, including macro pores as designed and micropores within the lines that compose the structure. This microporosity provides the auxetic structure with additional permeability for enhanced biological performance.

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the reference list. For example, Ref. [1] refers to the 1^{st} reference in the list. All references cited and discussed in this specification are incorporated herein by reference in their entirety and to the same extent as if each reference was individually incorporated by reference.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

1. An apparatus comprising:
a 3D auxetic structure formed of a periodic network of orthogonally patterned regions, including a first orthogonally patterned region and a second orthogonally patterned region, the first orthogonally patterned region being coupled to the second orthogonally patterned region, each comprising:
a first longitudinal beam having a first length,
a second longitudinal beam fixably attached to the first longitudinal beam at a first hinge region, the second longitudinal beam having a second length and is orthogonally disposed with respect to the first longitudinal beam, wherein the first length is different from the second length; and
a third longitudinal beam fixably attached to the first longitudinal beam at a second hinge region, the third longitudinal beam having a third length and is orthogonally disposed with respect to the first longitudinal beam and the second longitudinal beam, wherein the third length is different from the first length and second length,
wherein the first hinge region, the first longitudinal beam, the second hinge region, and the third longitudinal beam are configured to bend under load applied in a direction non-parallel to the longitudinal axis of the first longitudinal beam.

2. The apparatus of claim 1, wherein the first orthogonally patterned region and the second orthogonally patterned region each forms an auxetic unit-cell having symmetrical arrangements of orthogonally patterned beams on each orthogonal plane.

3. The apparatus of claim 1, wherein the first hinge region, the first longitudinal beam, the second hinge region, and the third longitudinal beam are configured to bend under the load as a compression load from a bending behavior of the first longitudinal beam and third longitudinal beam when the first hinge region and the second hinge region bend under the same compression load.

4. The apparatus of claim 1, wherein the first longitudinal beam has a constant first cross-section area, wherein the second longitudinal beam has a constant second cross-section area, wherein the third longitudinal beam has a constant third cross-section area, wherein the first cross-section area is the same or different as the second cross-section area, and wherein the second cross-section area is the same or different as the third cross-section area.

5. (canceled)

6. (canceled)

7. The apparatus of claim 1, wherein the auxetic structure is a low-density structure having a define-able void fraction between 60% and 95%.

8. The apparatus of claim 1, wherein the first longitudinal beam of the first orthogonally patterned region has a first end and second end, and wherein the first longitudinal beam of the second orthogonally patterned region is fixably coupled to the first longitudinal beam of the first orthogonally patterned region at the first end or in between the first end and second end.

9. (canceled)

10. The apparatus of claim 1, further comprising:
a third orthogonally patterned region coupled to the second orthogonally patterned region, the third orthogonally patterned region comprising:
a fourth longitudinal beam having a fourth length,
a fifth longitudinal beam fixably attached to the fourth longitudinal beam at a third hinge region, the fifth longitudinal beam having a fifth length and is orthogonally disposed with respect to the fourth longitudinal beam, wherein the fourth length is different from the fifth length; and
a sixth longitudinal beam fixably attached to the fourth longitudinal beam at a fourth hinge region, the sixth longitudinal beam having a sixth length and is orthogonally disposed with respect to the fourth longitudinal beam and the fifth longitudinal beam, wherein the sixth length is different from the fourth length and fifth length,
wherein the third hinge region, the fourth longitudinal beam, the fourth hinge region, and the sixth longitudinal beam are configured to bend under the load applied in the direction non-parallel to the axis of the fifth longitudinal beam.

11. The apparatus of claim 10, wherein the third orthogonally patterned region is disposed proximal to the first orthogonally patterned region such that the fourth longitudinal beam of the third orthogonally patterned region is parallel to the first longitudinal beam of the first orthogonally patterned region.

12. The apparatus of claim 1, wherein the auxetic structure comprises a biocompatible material, a polymeric material, or a biodegradable material.

13. (canceled)

14. (canceled)

15. The apparatus of claim 1, comprising one or more bioactive materials; with or without growth factor reagents and/or therapeutics.

16. The apparatus of claim 1, wherein the first longitudinal beam is fabricated with microscale voids.

17. The apparatus of claim 1, wherein the auxetic structure is fabricated via additive manufacturing.

18. The apparatus of claim 1, wherein the orthogonally patterned regions include a repeating L-shape or C-shape structure in the first planar direction and a second planar direction orthogonal to the first planar direction.

19. (canceled)

20. (canceled)

21. The apparatus of claim 1, wherein the orthogonally patterned regions include a repeating L-shape structure coupled to a repeating C-shape structure in a first planar direction and a second planar direction orthogonal to the first planar direction.

22. The apparatus of claim 1, wherein the apparatus is configured as a tissue or organ implant.

23. The apparatus of claim 22, wherein the apparatus is configured as an implant for one of: breast reconstruction, facial reconstruction, calcaneal fat pad reconstruction, buttock reconstruction, or brain tissue defect reconstruction.

24. The apparatus of claim 1, wherein the apparatus is configured for use as an in vitro tissue model.

25. The apparatus of claim 24, wherein the in vitro tissue model comprises hydrogel-based biomaterials incapsulating cardiac cells and relevant bioactive materials, growth factors, or drugs.

26. The apparatus of claim 24, wherein the apparatus is configured as an in vitro model of one of: cardiac tissue, adipose tissue, liver tissue, kidney tissue, neuronal tissue, cartilage tissue.

27. The apparatus of claim 24, wherein the in vitro tissue model comprises one or more of: cardiac cells, adipose cells, adipose derived stem cells, hepatocytes, human skeletal muscle cells, induced pluripotent stem cells, chondrocytes.

28. The apparatus of claim 24, wherein the in vitro tissue model is configured for of one or more of: biological assessment, detecting and modeling of diseases and disorders, and drug discovery and screening, lab-grown food testing.

29. The apparatus of claim 1, wherein the auxetic structure comprises a framework for a hydrogel.

30. The apparatus of claim 1, wherein the apparatus is configured to conform to patient-specific anatomy.

31. An apparatus, comprising:
a network of auxetic structure unit-cells configured to be utilized for purposes of adipose tissue reconstruction or breast reconstruction.

32. (canceled)

33. (canceled)

34. A method comprising:
forming, via additive manufacturing, the apparatus of claim 1.

35. The apparatus of claim 1, wherein the apparatus is a computer model for tissue modeling in a virtual environment.
